(19) United States
(12) Patent Application Publication
(10) Pub. No.: US 2009/0054694 A1
(43) Pub. Date: Feb. 26, 2009

(54) Chemical production processes, systems, and catalyst compositions

(76) Inventors: Thomas H. Peterson, Midland, IL (US); Johnathan E. Holladay, Kennewick, WA (US); Alan H. Zacher, Pasco, WA (US); Michel J. Gray, Pasco, WA (US); Todd A. Werpy, West Richland, WA (US); James F. White, Richland, WA (US)

Correspondence Address: WELLS ST. JOHN P.S., 601 WEST FIRST AVENUE, SUITE 1300, SPOKANE, WA, 99201-3828, US

(21) Appl. No.: 11/895,593

(22) Filed: Aug. 24, 2007

(51) Int. Cl. C07C 45/29

(52) U.S. Cl. 568/361; 568/485

(57) ABSTRACT

Chemical production processes are provided that include exposing a reactant composition to a catalyst composition to form a product composition, with the reactant composition including a multihydric alcohol compound and the catalyst composition being effective to dehydrate at least a portion of the multihydric alcohol compound. Embodiments of the process provide that the reactant composition is exposed to the catalyst composition for less than about 0.25 seconds and/or that the catalyst is maintained at a temperature of from about 280° C. to about 320° C. Processes utilizing a reactant composition including a multihydric alcohol compound and an inert compound are also provided.

### RELATED PATENT DATA

This application is a continuation in part of U.S. patent application Ser. No. 11,895,362, entitled Chemical Production Processes, Systems, and Catalyst Compositions by Peterson et. al. which was filed on Aug. 24, 2007; Ser. No. 11,895,414, entitled Chemical Production Processes, Systems, and Catalyst Compositions by Peterson et. al. which was filed on Aug. 24, 2007; Ser. No. 11,895,592, entitled Chemical Production Processes, Systems, and Catalyst Compositions by Peterson et. al. which was filed on Aug. 24, 2007; the entirety of all are incorporated by reference herein.

### TECHNICAL FIELD

The present disclosure relates to chemical production processes, systems, and catalyst compositions.

### BACKGROUND OF THE DISCLOSURE

For nearly a century, scientists have struggled with the efficient dehydration of multihydric alcohol compounds. For example, the dehydration of glycerol to acrolein, acetol, and glycerol oligomers was first reported by Nef in 1904. When compositions were heated to temperatures between 430° C. and 450° C., carbonaceous materials and a distillate were produced that contained acrolein, acetol, water, and formaldehyde among other products. Over the next 100 years, occasional reports of catalyzed conversions of glycerol had been communicated targeting conversion to acrolein and acetol directly. As an example, the condensation of glycerol to di-, tri-, and oligoglycerol ethers had been effected with basic catalysts. However, when acidic catalysts were employed, acrolein is formed as a major by-product. As another example, acrolein has also been reported as a product of castor oil hydrolysis and cracking. Conversion of multihydric alcohol compounds has been performed in the temperature range of 250° C. to 400° C., utilizing phosphate or sulfate acid or acid salt as a catalyst. However, clays, zeolites, CO₂ or autoionization of supercritical H₂O has been shown to effect dehydration with the yields of acrolein rarely exceeding 70%.

While examples of glycerol to acrolein transformation do exist, they are relatively few in number. As recently as 1994, U.S., Japanese, and European patents have been awarded describing the conversion of glycerol to acrolein and acrolein hydrogenation to a mixture of isomeric propanediols. In 1998, platinum bisphosphine complexes were used in the presence of strong acids and syn gas to carry out the conversion of glycerol to acrolein in 80% yield. More recently, glycerol dehydration in subcritical water catalyzed by ZnSO₄ in a staged reactor process has been utilized to convert glycerol to acrolein and acrylic acid. To date, processes having high selectivity and commercial viability are still not known for the conversion of glycerol to acrolein.

### SUMMARY OF THE DISCLOSURE

Chemical production processes are provided that include exposing a reactant composition to a catalyst composition to form a product composition, with the reactant composition including a multihydric alcohol compound and the catalyst composition being effective to dehydrate at least a portion of the multihydric alcohol compound. Embodiments of the process provide that the reactant composition is exposed to the catalyst composition for less than about 0.25 seconds and/or that the catalyst is maintained at a temperature of from about 280° C. to about 320° C.

Chemical production processes are also provided that include exposing a reactant composition to a catalyst composition to form a product composition, with the reactant composition comprises a multihydric alcohol compound and an inert compound.

### BRIEF DESCRIPTION OF THE DRAWINGS

Preferred embodiments of the disclosure are described below with reference to the following accompanying drawings.

FIG. 1 is a chemical production system according to an embodiment of the disclosure.

FIG. 2 is a plot of data acquired utilizing an embodiment of the disclosure.

FIG. 3 is a plot of data acquired utilizing an embodiment of the disclosure.

FIG. 4 is a plot of data acquired utilizing an embodiment of the disclosure.

FIG. 5 is a chemical production system according to another embodiment of the disclosure.

### DESCRIPTION

This disclosure is submitted in furtherance of the constitutional purposes of the U.S. Patent Laws “to promote the progress of science and useful arts” (Article 1, Section 8).

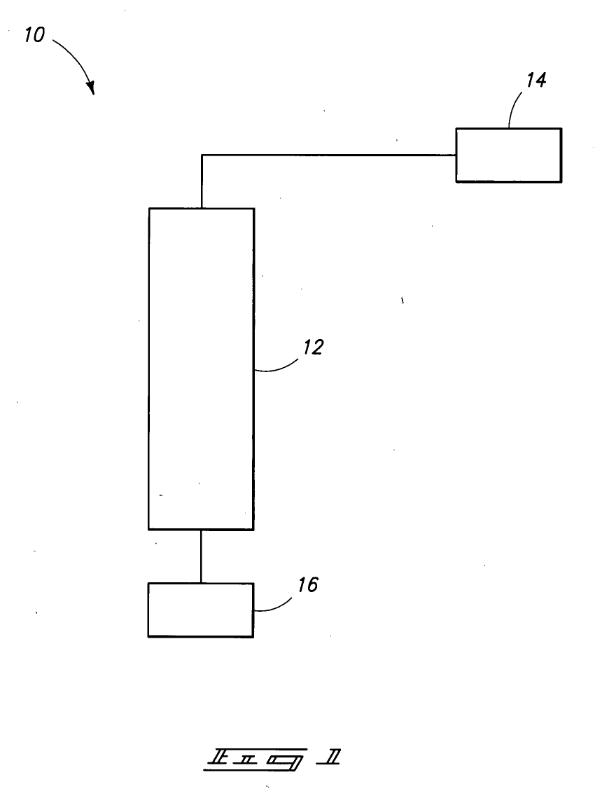

The chemical production processes of the present disclosure will be described with reference to FIGS. 1-5. Referring first to FIG. 1, a chemical production process system 10 is shown that includes a reactor 12 coupled to both a reactant reservoir 14 and a product reservoir 16. In accordance with the present disclosure, reactant reservoir 14 can be coupled to reactor 12 utilizing conduits that facilitate the flow of reactant from reactant reservoir 14 to reactor 12. This flow can be facilitated utilizing pressure differentials between reactant reservoir 14 and reactor 12. For example, these pressure differentials can be facilitated utilizing pumps to provide a pressure differential between reactant reservoir 14 and reactor 12. The reactant within reactant reservoir 14 can be a multihydric alcohol compound. An example multihydric alcohol compound can include the compound glycerol, which when dehydrated can result in a product composition that includes one or both of acrolein and/or acetol, for example.

Reactor 12 can include a housing that can be configured to house a catalyst and be utilized to facilitate the exposure of the reactant within reactant reservoir 14 to catalyst within reactor 12. Reactor 12 can define a volume containing the catalyst. When reactor 12 is supplied with catalyst and/or packing a void volume exists within reactor 12. When reactant is supplied to reactor 12 this void volume can be occupied by the reactant. The amount of reactant used to consume the void volume can be used to calculate the time reactant is exposed to catalyst, for example the residence time and/or contact time. Reactor 12 can be jacketed or can be configured as a fluidized bed reactor, for example.

The product composition provided to product reservoir 16 can be a dehydration product of the multihydric alcohol compound such as a carbonyl compound. The pressure differential apparatus used to facilitate the transfer of reactant from reactant reservoir 14 can also be utilized to provide product from reactor 12 to product reservoir 16. In accordance with an example embodiment, system 10 can be configured to expose a multihydric alcohol compound such as glycerol from reservoir 14 to a catalyst composition within reactor 12 to form a product composition including one or both of acrolein and acetol.

The catalyst can be a catalyst that is effective to dehydrate at least a portion of a multihydric compound. The catalyst can include those disclosed in the prior art as well as the novel dehydration catalysts of this disclosure. The catalyst can be supported and/or unsupported catalyst, for example. Unsupported catalysts can be referred to as bulk catalysts.

According to an example embodiment, the chemical production process can include exposing the reactant composition to a catalyst composition for less than about 0.25 seconds. According to other embodiments, the reaction composition can be exposed to the catalyst composition for less than about 0.14 seconds. As another example, the reaction composition can be exposed to the catalyst composition for from about 0.07 seconds to about 0.25 seconds.

Where the multihydric compound is glycerol, the product composition can include both acrolein and acetol. Utilizing the contact times above, for every mole of glycerol exposed to the catalyst, at least about 0.1 moles of product composition can be formed. In accordance with another example, every mole of glycerol can form from about 0.1 to about 0.99 moles of product composition. Further, utilizing the contact times above, the product composition can include acrolein and acetol at a ratio of at least about 3:2 and/or from about 3:2 to about 10:1. Many catalysts can be used to accomplish dehydration of the reactant at these contact times.

The catalyst can be solid support materials including silica, silica alumina zirconia, and acidic fluoride-treated alumina, for example. More particularly, the catalyst can be a solid support composition such as one or more of F—Al₂O₃, ZrO₂—CO₂, SiO₂—Al₂O₃—CO₂, SiO₂—Al₂O₃, Alundum, and Silica such as Ludox AS-30.

Table 1 below is an indication of the results of the utilization of these solid supports at various reactor temperatures.

TABLE 1
Examination of support materials for glycerol dehydration at 300-335° C.

| | | Mass | Carbon | | Acrolein | | |
|---|---|---|---|---|---|---|---|
| | T | Balance | Balance | Conv. | Yield | Acrolein | Acetol |
| Catalyst Description | (C.) | (%) | (%) | (%) | (%) | Selectivity | Selectivity |
| fluoride treated Al₂O₃ | 300 | 107.17 | 83.52 | 51.16 | 23.66 | 0.462 | 0.215 |
| ZrO₂—CO₂ | | 87.81 | 99.26 | 3.70 | 1.23 | 0.332 | 0.469 |
| SiO₂—Al₂O₃—CO₂ | | 103.26 | 59.71 | 93.89 | 37.96 | 0.404 | 0.167 |
| SiO₂—Al₂O₃ | | 104.94 | 104.67 | 41.38 | 37.32 | 0.902 | 0.211 |
| Alundum | | 70.36 | 70.40 | 29.68 | 0.08 | 0.003 | 0.000 |
| Silica derived from | | 99.23 | 94.63 | 5.37 | 0.00 | 0.000 | 0.000 |
| Ludox AS-30 | | | | | | | |
| fluoride treated Al₂O₃ | 320 | 101.69 | 62.16 | 98.98 | 43.56 | 0.440 | 0.178 |
| ZrO₂—CO₂ | | 98.98 | 93.69 | 12.63 | 2.3 | 0.182 | 0.319 |
| SiO₂—Al₂O₃—CO₂ | | 103.87 | 62.38 | 99.34 | 44.17 | 0.445 | 0.177 |
| SiO₂—Al₂O₃ | | 108.92 | 94.60 | 81.31 | 60.73 | 0.747 | 0.187 |
| Alundum | | 100.75 | 98.32 | 1.68 | 0.00 | 0.000 | 0.000 |
| ZrO₂—CO₂ | 335 | 87.20 | 69.77 | 48.39 | 6.30 | 0.130 | 0.245 |
| SiO₂—Al₂O₃ | | 97.60 | 56.84 | 94.92 | 39.61 | 0.417 | 0.128 |

The qualitative and quantitative data of Table 1 above as well as all remaining data of the disclosure can be acquired utilizing gas and liquid chromatography techniques. For example, gas chromatographic analyses can be performed utilizing a Shimadzu GC-2010 Gas Chromatograph (GC) equipped with a Flame Ionization Detection (FID) operating at 280° C., and an AOC-20 autosampler, and employing GC Solutions Software. A DB-WAX (J & W Scientific) capillary column (30 m×0.32 mm I.D.×0.25 μm film thickness) can be employed utilizing helium as carrier gas at a 2.61 mL/min flow rate. Injections of 1 μL utilizing a 25:1 split ratio can be made with the injector port maintained at 250° C. Oven temperature programming can utilize an initial temperature of 40° C. with a hold for 5 minutes followed by a 10° C./min ramp to 245° C. and a hold at the final temperature for 4.5 minutes. Calibrations can be performed on a periodic or monthly basis using known standard solutions for glycerol, acrolein, and acetol. Calibrations can take place using a series of five standard solutions prepared by serial dilution to determine the linear response for each compound, and acceptance of each curve determined if the linear response had an R value of greater than 0.99.

Liquid chromatographic analyses can be carried out on a Waters LC system incorporating a Waters 515 pump, Waters 2410 Refractive Index Detector (RID), and a Waters 717 plus Autosampler for sample introduction. Analyses can be performed utilizing Empower Pro Software. Separations of 10 μL injections can be effected on an Aminex HPX-87H Organic Acid Analysis column operated at 35° C. and employing a 0.005M H₂SO₄ as the eluent with a flow rate of 0.55 mL/min. Total run times of 45 minutes were sufficient to elute all compounds of interest. Calibration curves can be prepared as described for GC calibrations and using the same set of standard solutions used for GC calibration.

Referring to product reservoir 16 of system 10, upon exiting reactor 12, product can be acquired by time collection of reactor 12 effluent in a known quantity of a chilled scrub solution containing 1 wt % n-BuOH with mass balances for a given reactor run determined by a ratio of collected effluent mass to expected mass based on feed rate and run time. For example, two small aliquots can be removed and diluted to concentrations appropriate for GC and LC analyses. The diluted samples can then be analyzed as described previously and wt % compositions determined from calibrated detector responses used to determine absolute compositions of the collected effluent. The use of known quantities of n-BuOH in the scrub solutions can permit a primary check of the analytical sampling technique. Reported values for conversion, yield, selectivity, and carbon balance present averages of those values determined by both GC and LC analyses. Glycerol conversion can be calculated by the differences between calculated quantity of glycerol feed (based on feed rate and run time) and the quantity of glycerol collected in the reactor effluent and may be uncertain when mass balances are not satisfactory. As such, values exclude any experimental runs that did not provide mass balances between 90% and 100%. Product yields can be calculated by the ratio of quantity of product formed to the quantity of glycerol. Product selectivities can be calculated from the quantity of product formed divided by the quantity of glycerol converted. Carbon balances can be calculated from the sum of the molar quantities of glycerol, acrolein, and acetol components divided by the molar quantity of glycerol fed. Liquid Chromatographic techniques can permit the quantification of formic acid and acetic acid by-products. However, since their combined quantity rarely exceeded 3%, their presence was not included in carbon balance determination.

According to another embodiment, the catalyst can include a metal phosphorous composition. The metal phosphorous composition can comprise one or more of Si and Ti, for example. According to exemplary embodiments, the catalyst composition can comprise a solid substrate, comprising one or more of SiO₂, SiO₂—Al₂O₃, and TiO₂. The solid substrate can be impregnated with a phosphoric acid. The phosphoric acid can be from about 8% (wt./wt.) to about 35% (wt./wt.) of the catalyst composition. According to a particular embodiment, the catalyst composition can comprise a solid substrate including SiO₂—Al₂O₃, and the solid substrate can be impregnated with a phosphoric acid from about 8% (wt./wt.) to about 30% (wt./wt.) of the catalyst composition. The catalyst composition can also include a solid substrate comprising SiO₂, and the solid substrate can be impregnated with phosphoric acid to a level of from about 29% (wt./wt.) to about 35% (wt./wt.) of the catalyst composition.

Impregnation of these metal phosphorous catalysts can be performed by incipient wetness of an appropriate quantity of 85 wt % H₃PO₄ in deionized water to give the desired loading during impregnation. Following impregnation, the catalysts can be dried at 100° C. and used without further treatment. The performance of these catalysts is shown below in Table 2.

TABLE 2
Performance of supported phosphoric acid catalysts for glycerol
dehydration at 300° C.

| | | Contact | Carbon | | Acrolein | | |
|---|---|---|---|---|---|---|---|
| Wt % | Catalyst | Time | Balance | Conv. | Yield | Acrolein | Acetol |
| H₃PO4 | Support | (s) | (%) | (%) | (%) | Selectivity | Selectivity |
| 8 | SiO₂—Al₂O₃ | 0.25 | 74.49 | 81.47 | 48.28 | 0.594 | 0.095 |
| 20 | SiO₂—Al₂O₃ | 0.11 | 91.56 | 46.26 | 33.18 | 0.717 | 0.100 |
| 20 | SiO₂—Al₂O₃ | 0.06 | 99.72 | 17.26 | 14.80 | 0.857 | 0.126 |
| 25 | Al₂O₃ | 0.30 | 49.14 | 95.23 | 34.48 | 0.362 | 0.104 |
| 25 | Al₂O₃ | 0.30 | 41.76 | 94.68 | 27.98 | 0.296 | 0.089 |
| 30 | SiO₂—Al₂O₃ | 0.21 | 79.58 | 41.56 | 17.83 | 0.429 | 0.080 |
| 35 | SiO₂ | 0.12 | 84.29 | 83.49 | 58.44 | 0.700 | 0.112 |
| 35 | SiO₂ | 0.07 | 95.52 | 47.78 | 37.75 | 0.790 | 0.116 |
| 29* | SiO₂ | 0.24 | 93.83 | 99.17 | 80.48 | 0.812 | 0.126 |

*utilized (NH₄)₂HPO₄ as a catalyst precursor calcined at 350° C.

Contact times shorter than 4 seconds can be sufficient to effect dehydration of glycerol at 300° C. In accordance with some embodiments, greater than 90% glycerol dehydration can be realized with contact times of 300 milliseconds or less. Reactions performed at less than about 280° C. can form coke as a by-product. For contact times greater than 200 milliseconds, conversion can range between 40% and 99%. Selectivities for acrolein and acetol using solid phosphoric acid catalysts can range from 70% and 10%, respectively.

In accordance with another embodiment, the catalyst can include a metal phosphate composition. The metal phosphate composition can include one or more elements from groups 2-7 and 9-12 of the periodic table of elements. The metal phosphate composition, for example, can include one or more of Cr, Mn, Fe, Co, Ni, Zn, La, Ca, Sr, Ba, Mo, Al, B, and Ru. The metal phosphate composition can be in the form of a metal dihydrogen phosphate, a metal hydrogen phosphate, or a metal phosphate. The metal phosphate composition can also include phosphoric acid as well. According to exemplary embodiments, the metal phosphate composition can be M_{0.33}H_{2.33}PO₄ with M being one or more of Cr, Mn, Fe, Ru, Co, Ni, Zn, Ba, or La.

These metal phosphate composition catalysts can be prepared, for example, in a 200 mL beaker charged with metal nitrite and dissolved with a minimal amount of deionized water, for example. A 25 wt % solution of (NH₄)₂HPO₄ in deionized water can be prepared and an appropriate quantity of solution transferred to a small beaker. Ludox AS-40 can be placed in a graduated cylinder. With stirring, the ammonium phosphate and Ludox solutions can be poured concurrently into the nitrate solution, resulting in precipitation of metal phosphates. Stirring can be continued overnight and, depending on composition, sometimes results in a partial gelation. Excess water can then be removed on a rotary evaporator at 40 torr while employing a bath temperature of 60° C. The dried solids can be calcined in air for about 6 hours at 350° C. The calcined solids can then be crushed and sieved to a size appropriate for the reactor employed.

Tables 3 and 4 below are exemplary of the data acquired utilizing these metal phosphate composition catalysts.

TABLE 3
Performance of supported metal phosphate-phosphoric acid catalysts for
glycerol dehydration at 300° C.

| | | Mass | Carbon | | Acrolein | | |
|---|---|---|---|---|---|---|---|
| | Contact Time | Balance | Balance | | Yield | Acrolein | Acetol |
| Metal | (sec) | (%) | (%) | Conv. (%) | (%) | Selectivity | Selectivity |
| Ba | 0.25 | 97.06 | 98.67 | 99.78 | 84.28 | 0.845 | 0.142 |
| Cr | 0.24 | 90.69 | 84.33 | 30.41 | 12.38 | 0.407 | 0.078 |
| Mn | 0.23 | 97.88 | 93.35 | 99.88 | 80.43 | 0.805 | 0.128 |
| Fe | 0.25 | 102.60 | 96.97 | 99.49 | 83.07 | 0.835 | 0.135 |
| Co | 0.21 | 100.96 | 96.79 | 99.60 | 82.00 | 0.823 | 0.145 |
| Ni | 0.24 | 94.39 | 86.13 | 92.40 | 68.08 | 0.737 | 0.113 |
| Zn | 0.24 | 94.70 | 91.83 | 99.86 | 82.07 | 0.822 | 0.096 |
| La | 0.24 | 90.83 | 75 85 | 86.77 | 53.36 | 0.615 | 0.107 |
| Ru | 0.25 | 98.41 | 63.19 | 100.00 | 43.69 | 0.437 | 0.195 |
| Mo | 0.28 | 95.68 | 44.26 | 80.42 | 21.66 | 0.269 | 0.038 |

TABLE 4
Performance of supported metal phosphate-phosphoric acid catalysts for
glycerol dehydration at 320° C.

| | | Mass | Carbon | | Acrolein | | |
|---|---|---|---|---|---|---|---|
| | Contact Time | Balance | Balance | | Yield | Acrolein | Acetol |
| Metal | (sec) | (%) | (%) | Conv. (%) | (%) | Selectivity | Selectivity |
| Ba | 0.25 | 81.17 | 74.29 | 93.46 | 59.56 | 0.637 | 0.088 |
| Mn | 0.24 | 90.69 | 89.28 | 95.01 | 74.88 | 0.79 | 0.10 |
| Fe | 0.23 | 86.71 | 82.28 | 24.75 | 6.06 | 0.245 | 0.039 |
| Zn | 0.24 | 95.68 | 90.85 | 99.58 | 79.69 | 0.800 | 0.108 |
| Co | 0.25 | 89.86 | 91.40 | 98.73 | 79.51 | 0.81 | 0.11 |
| Mo | 0.29 | 97.98 | 48.89 | 87.30 | 31.69 | 0.363 | 0.052 |
| Ni | 0.24 | 96.26 | 87.45 | 96.44 | 73.81 | 0.765 | 0.105 |

Quantitative glycerol conversion can be effected in catalysts using contact times of less than 250 milliseconds. Acrolein selectivities typically exceeded 80% with catalysts incorporating metals of the middle transition series and acetol selectivities consistently ranged between 10% and 20%. Coke formation can be observed with the most active catalysts.

Catalyst compositions can affect glycerol conversion to 50% or greater. For example, when 5% silica alumina is utilized at 300° C., a 41% glycerol conversion can be realized.

In accordance with another embodiment, the catalyst can include elements of group 5 and 6 of the periodic table of elements, for example such as the polyoxometallates of these elements. More particularly, the catalyst can include Nb, Mo, and/or W. The catalyst can be hydrated or oxidized. For example, the catalyst can be hydrated nobia. The catalyst can include tungstic acid and/or phosphotungstic acid. The catalyst can include phosphomolybdic acid. The catalyst can be supported with a silica support. Prior to exposing reactant to the catalyst, the catalyst can be exposed to carrier composition such as CO₂.

The catalyst can be prepared in a jar by placing 6 g of tungstic acid, for example, with a stir bar in the jar. The acid can be slurried with 20 mL of H₂O and approximately 15 mL of 15M aqueous ammonia (solution pH=12.5). Colloidal silica (58 g, 42 mL) can then be added to the jar with vigorous stirring resulting in precipitation. After stirring for 2 hours, the solution can be concentrated to dryness on a rotary evaporator with a bath temperature of 50° C. and operating at a pressure of 40 torr. The solid can be transferred to a porcelain crucible and calcined for 6 hours at 330° C. to provide a powder. Subsequently, the powder can be crushed and sieved to 45×100 mesh, for example.

Hydrated forms of metal oxides of the catalyst can demonstrate acidic behavior, and the overall acidity can be controlled by the degree of hydration. In some cases, (niobia, for example) the degree of hydration can itself be controlled by the pre-treatment temperature of the oxide. Thus pretreatment can provide a vehicle for tuning the acidity of a material.

Pelleted niobia can be calcined for periods of 6 hours at temperatures of 200° C., 300° C., 400° C., and 500° C. The calcined materials can then be crushed and sieved to a size appropriate for the reactor and evaluated at temperatures between 280° C. and 320° C. employing residence times of 300 milliseconds or less. Results for catalysts, which are tabulated as averages of multiple runs, are shown in Tables 5 and 6 below.

TABLE 5
Performance of hydrated niobia for glycerol dehydration at 300° C.

| Calcination | | Carbon | | Acrolein | | Acetol |
|---|---|---|---|---|---|---|
| Temperature | Sup- | Balance | Conv. | Yield | Acrolein | Selec- |
| (° C.) | port | (%) | (%) | (%) | Selectivity | tivity |
| 200 | bulk | 56.70 | 85.88 | 34.85 | 0.41 | 0.09 |
| 300 | bulk | 66.10 | 89.48 | 47.39 | 0.530 | 0.022 |
| 400 | bulk | 50.52 | 95.43 | 38.94 | 0.41 | 0.07 |
| 400 | silica | 93.45 | 55.28 | 38.89 | 0.72 | 0.19 |
| 500 | bulk | 53.56 | 3.32 | 5.28 | 0.000 | 0.000 |

TABLE 6
Performance of hydrated niobia for glycerol dehydration at 320° C.

| Calcination | | Carbon | | Acrolein | | Acetol |
|---|---|---|---|---|---|---|
| Temperature | Sup- | Balance | Conv. | Yield | Acrolein | Selec- |
| (° C.) | port | (%) | (%) | (%) | Selectivity | tivity |
| 200 | bulk | 52.35 | 99.64 | 44.23 | 0.44 | 0.08 |
| 300 | bulk | 82.65 | 97.78 | 70.90 | 0.725 | 0.097 |
| 400 | bulk | 69.26 | 99.09 | 56.40 | 0.569 | 0.121 |
| 400 | silica | 88.98 | 75.02 | 50.58 | 0.67 | 0.18 |
| 500 | bulk | 46.95 | 22.65 | 14.25 | 0.629 | 0.236 |

For catalyst calcined at 500° C., conversion was reduced, consistent with reduced acidity. The differences between niobia samples calcined at 500° C. and lower temperatures indicate that niobia rehydration to more acidic forms, even under hydrothermal conditions, may not occur. For runs at 300° C. and 320° C., the niobia samples calcined at 200° C. can provide low yields and carbon balances. Niobia dispersion onto an inert support, (Tables 5 and 6, silica entry) can result in lower activity but increased selectivity. Both observations can be consistent with reduced total acidity in the prepared catalysts which lead to lower rates for both catalytic dehydration and catalyst-promoted product decomposition.

Commercial samples of tungstic and phosphotungstic acid can be dissolved in water and dispersed onto colloidal silica to prepare the catalyst as well. The supported catalysts can be evaluated for glycerol dehydration at 300° C. and 320° C. employing residence times of 140 and 250 milliseconds. Data are tabulated below in Tables 7 and 8.

TABLE 7
Performance of tungsten acids for glycerol dehydration at 300° C.

| | Con- | | | | | |
|---|---|---|---|---|---|---|
| Catalyst | tact | Carbon | | Acrolein | | Acetol |
| (20 w % on | Time | Balance | Conv. | Yield | Acrolein | Selec- |
| SiO₂) | (sec) | (%) | (%) | (%) | Selectivity | tivity |
| H₂WO₄ | .014 | 95.87 | 41.1 | 33.43 | 0.813 | 0.086 |
| H₃O₄₄W₁₂P | | 94.49 | 35.65 | 28.12 | 0.785 | 0.086 |
| H₃Mo₁₂O₄₀P | | 32.38 | 38.1 | 0.99 | 0.026 | 0.011 |
| H₂WO₄ | .025 | 89.85 | 71.45 | 55.55 | 0.777 | 0.081 |
| H₃O₄₄W₁₂P | | 84.47 | 67.52 | 46.90 | 0.692 | 0.074 |
| Mo₁₂O₄₀P | | 33.16 | 36.67 | 1.34 | 0.036 | 0.017 |

TABLE 8
Performance of tungsten acids for glycerol dehydration at 320° C.

| | Con- | | | | | |
|---|---|---|---|---|---|---|
| Catalyst | tact | Carbon | | Acrolein | | Acetol |
| (20 w % on | Time | Balance | Conv. | Yield | Acrolein | Selec- |
| silica) | (sec) | (%) | (%) | (%) | Selectivity | tivity |
| H₂WO₄ | .013 | 93.72 | 50.16 | 38.61 | .77 | 0.105 |
| H₃O₄₄W₁₂P | | 95.95 | 31.85 | 24.37 | .783 | 0.111 |
| H₂WO₄ | .024 | 91.57 | 63.09 | 47.94 | .76 | 0.107 |
| H₃O₄₄W₁₂P | | 87.48 | 54.58 | 36.93 | .665 | 0.091 |

At 300° C., both tungsten- and molybdenum-based acids can be active, with tungsten acids showing acrolein selectivities in the range of 0.69-0.81. At shorter residence times selectivities can increase. Similar trends can be observed for experiments conducted at 320° C., though overall selectivities can be lower at this temperature.

Catalyst compositions incorporating phosphoric acid, metal phosphates and niobia can be prepared and used for glycerol dehydration. Results are presented in Table 9 below.

TABLE 9
Performance of mixed catalysts for glycerol dehydration at 300° C.

| | Contact | Carbon | | Acrolein | | |
|---|---|---|---|---|---|---|
| | Time | Balance | Conv. | Yield | Acrolein | Acetol |
| Catalyst (on SiO₂) | (sec) | (%) | (%) | (%) | Selectivity | Selectivity |
| 5% Mn/Nb₂O₅ | 0.15 | 94.66 | 16.78 | 9 | 0.536 | 0.144 |
| 79% (5% Mn/Nb₂O₅) | | 99.72 | 12.72 | 9.61 | 0.769 | 0.223 |
| 5% Co/Nb₂O₅ | | 87.01 | 25.69 | 10.17 | 0.4 | 0.1 |
| 10% H₃PO₄/Nb₂O₅ | | 66.53 | 53.94 | 13.76 | 0.258 | 0.127 |
| 20% H₃PO₄/Nb₂O₅ | | 90.07 | 50.04 | 33.41 | 0.672 | 0.135 |
| 2.9% Mn77% Nb₂O₅ | | 100.65 | 12.29 | 10.25 | 0.842 | 0.219 |
| 8% H₃PO₄ | | | | | | |
| Mn/H₃PO₄/Nb₂O₅ | | 90.49 | 21.81 | 9.9 | 0.461 | 0.113 |
| 79% (5% Mn/Nb₂O₅) | 0.25 | 63.04 | 61.85 | 19.3 | 0.309 | 0.093 |
| 10% H₃PO₄/Nb₂O₅ | | 48.35 | 90.81 | 26.67 | 0.295 | 0.138 |
| 20% H₃PO₄/Nb₂O₅ | | 84.53 | 75.31 | 49.74 | 0.663 | 0.135 |
| 2.9% Mn77% Nb₂O₅ | | 90.04 | 41.72 | 25.29 | 0.607 | 0.156 |
| 8% H₃PO₄ | | | | | | |
| Mn/H₃PO₄/Nb₂O₅ | | 86.17 | 33.8 | 16.07 | 0.485 | 0.119 |

According to another embodiment, the catalyst within the reactor can be maintained at a temperature between from about 280° C. and 320° C. or from about 300° C. to about 320° C. The catalysts can be those disclosed herein as well as the dehydration catalysts previously disclosed. Utilizing these temperatures reactants of glycerol can used to form product compositions of acrolein and acetol. The product composition can be from about 0.1 moles to about 0.99 moles for every mole of reactant exposed to the catalyst. Further, a mole ratio of acrolein to acetol within the product composition can be at least 3:2 and/or from about 3:2 to about 10:1.

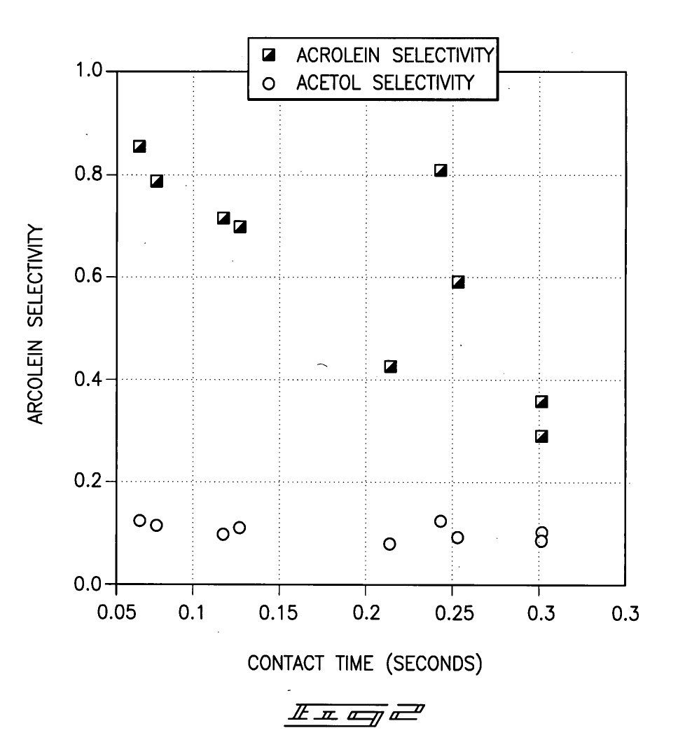

Significantly higher acrolein selectivities can be realized using short contact times. A plot of contact time versus acrolein selectivity and acetol selectivity is presented in FIG. 2. A strong linear correlation appears between contact time and acrolein selectivity. When considered in the context of an acrolein selectivity-carbon balance correlation, it can be that acrolein recovery is adversely affected by longer residence times and that acrolein decomposition or polymerization results in byproducts that are not a constituent of the carbon balance. This represents a first in that high activity catalysts operating at short contact-times may inherently lead to improved selectivities by reducing contact between products and catalyst.

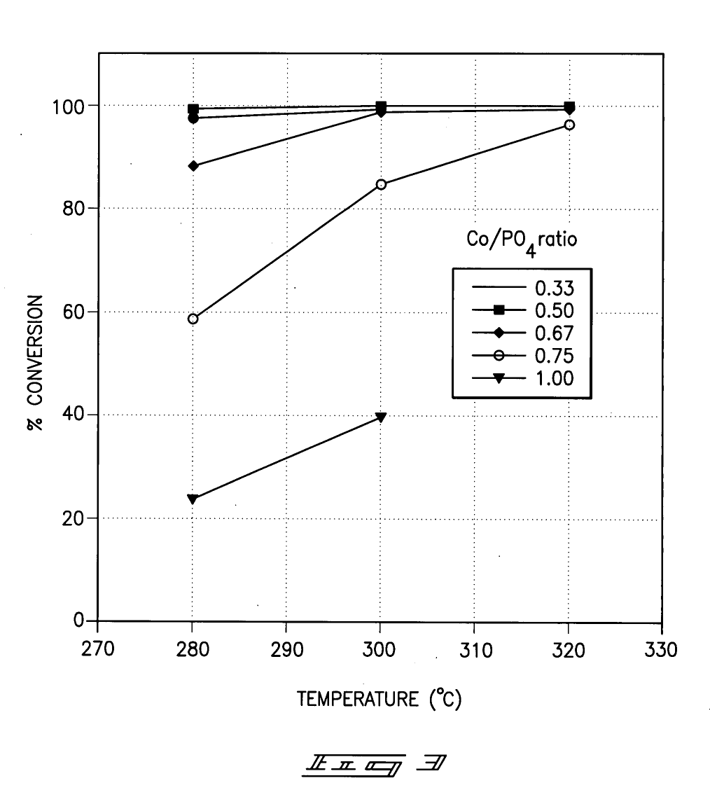

Referring to FIG. 1, a temperature within reactor 12 during exposure of reactant to these solid support materials can be in the range of about 300° C. to about 335° C. as well, using residence times of 250 to 300 milliseconds. Changing reaction temperature can increase activity such as conversion for specific active catalysts such as zirconia. As an example, data for a series of Co catalysts (varying Co/PO₄ ratio, see Tables 10-19) are illustrated graphically in FIG. 3.

TABLE 10
Effect of reaction temperature on selectivity and conversion for
Co_{0.33}H_{2.33}PO₄

| Feed | | Con- | | | | | |
|---|---|---|---|---|---|---|---|
| % | | tact | Carbon | | Acrolein | | Acetol |
| (wt./ | Temp | Time | Balance | Conv. | Yield | Acrolein | Selec- |
| wt.)^{†} | (° C.) | (sec) | (%) | (%) | (%) | Selectivity | tivity |
| 17 | 250 | 0.27 | 92.21 | 96.66 | 79.60 | 0.82 | 0.10 |
| 17 | 260 | 0.27 | 95.78 | 99.87 | 85.25 | 0.85 | 0.10 |
| 17 | 270 | 0.26 | 99.23 | 98.92 | 87.30 | 0.88 | 0.11 |
| 30 | 280 | 0.24 | 98.46 | 97.45 | 85.03 | 0.87 | 0.11 |
| 30 | 300 | 0.21 | 96.79 | 99.60 | 82.00 | 0.82 | 0.14 |
| 30 | 320 | 0.24 | 100.88 | 99.61 | 83.90 | 0.84 | 0.17 |

^{†}glycerol in water

Over two concentration ranges (which permit comparisons at constant conversion and contact time), selectivities for acetol can increase monotonically with increasing reaction temperature. The acrolein selectivity can appear to increase up to 270° C. and then begin decreasing up to 320° C. This can be consistent with the measured acrolein selectivity being very strongly correlated with carbon balance. Temperature can control both the intrinsic reaction kinetics (inherent mechanistic selectivities of a reaction) and product decomposition rates. An assessment of the propensity to form acrolein over acetol (intrinsic selectivity) can be made with extrapolation to 100% carbon balance (i.e., with the assumption of no product decomposition). To accomplish this assessment for example, the selectivity data for acrolein and acetol can be divided by the fractional carbon balances. The results of this example data manipulation are provided in Table 11 below.

TABLE 11
Effect of reaction temperature on intrinsic selectivities Co_{0.33}H_{2.33}PO₄

| Feed % | | Carbon | | | Intrinsic | Intrinsic |
|---|---|---|---|---|---|---|
| (wt./ | Temp | Balance | Acrolein | Acetol | Acrolein | Acetol |
| wt.)^{†} | (° C.) | (%) | Selectivity | Selectivity | Selectivity | Selectivity |
| 17 | 250 | 92.21 | 0.820 | 0.100 | 0.889 | 0.108 |
| 17 | 260 | 95.78 | 0.850 | 0.100 | 0.887 | 0.104 |
| 17 | 270 | 99.23 | 0.880 | 0.110 | 0.887 | 0.111 |
| 30 | 280 | 98.46 | 0.870 | 0.110 | 0.884 | 0.112 |
| 30 | 300 | 96.79 | 0.820 | 0.140 | 0.847 | 0.145 |
| 30 | 320 | 100.88 | 0.840 | 0.170 | 0.833 | 0.169 |

^{†}glycerol in water

Referring to Table 11, the acrolein selectivity is highest at the lower temperatures and decreases monotonically with increasing temperature with what appears to be the exact opposite trend observed for acetol. Similar results are obtained when data for the barium congener are treated analogously (Table 12).

TABLE 12
Effect of reaction temperature on intrinsic selectivities of
Ba_{0.33}H_{2.33}PO₄

| | Carbon | | | Intrinsic | Intrinsic |
|---|---|---|---|---|---|
| | Balance | Acrolein | Acetol | Acrolein | Acetol |
| T (° C.) | (%) | Selectivity | Selectivity | Selectivity | Selectivity |
| 250 | 91.86 | 0.794 | 0.101 | 0.865 | 0.110 |
| 260 | 90.82 | 0.803 | 0.104 | 0.885 | 0.115 |
| 270 | 97.30 | 0.855 | 0.118 | 0.879 | 0.121 |
| 280 | 74.29 | 0.637 | 0.088 | 0.858 | 0.118 |
| 300 | 98.67 | 0.845 | 0.142 | 0.856 | 0.144 |
| 320 | 103.62 | 0.89 | 0.15 | 0.855 | 0.145 |

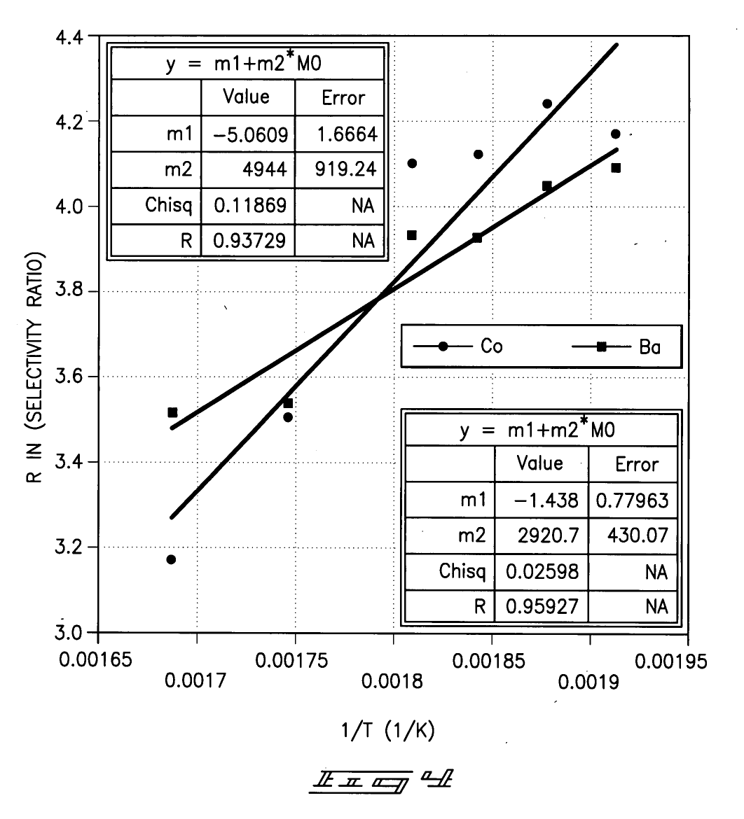

Referring to Table 12, the ratio of intrinsic selectivities can be related to the differences in the activation energies for the two transition states leading to acrolein and acetol from glycerol. By plotting R ln(ratio) versus (1/T), a linear relationship results, the slope of which represents the difference in transition state energies leading to acrolein and acetol. FIG. 4 shows such a plot using data from Tables 10 and 12. While some experimental scatter leads to somewhat less than desirable R-factors of 0.93 and 0.95 for Co and Ba, respectively, the data allow an estimation of the difference in activation energies for acrolein and acetol formation. For the barium catalyst, a value of 2.93±0.43 kcal mol⁻¹ is measured as the difference in the transition state energies of acrolein and acetol. For the cobalt catalyst, the calculated value is 4.94±0.92 kcal mol⁻¹.

TABLE 13
Calculated intrinsic selectivities for Co_{0.33}H_{2.33}PO₄ and
Ba_{0.33}H_{2.33}PO₄ as a function of temperature.

| | Intrinsic Acrolein | Intrinsic Acetol |
|---|---|---|
| T (° C.) | Selectivity (Co) | Selectivity (Ba) |
| 100 | 0.984 | 0.961 |
| 125 | 0.976 | 0.951 |
| 150 | 0.965 | 0.940 |
| 175 | 0.953 | 0.928 |
| 200 | 0.938 | 0.915 |
| 225 | 0.920 | 0.902 |
| 250 | 0.901 | 0.889 |
| 275 | 0.880 | 0.876 |
| 300 | 0.857 | 0.863 |
| 325 | 0.834 | 0.850 |
| 350 | 0.809 | 0.837 |
| 375 | 0.784 | 0.824 |
| 400 | 0.759 | 0.811 |
| 425 | 0.734 | 0.799 |
| 450 | 0.709 | 0.787 |

Referring to Table 13, catalysts can be prepared in the conventional manner by precipitation of metal phosphate in the presence of colloidal silica. The catalysts may maintain phosphate weight ratios of previous catalyst studies (phosphate:silica=0.29) but the metal-phosphate stoichiometry from the original 0.33:1 M:PO₄ ratio may be varied by increasing the metal nitrate precursor loading. Catalysts with Co:PO₄ ratios of 0.50, 0.67, 0.75 and 1.00 can be prepared in this manner and calcined at 350° C. for 6 hours prior to screening for comparison to the original 0.33:1 composition. Similarly, catalysts with Ba:PO₄ ratios of 0.50, 0.75, 1.00 and 1.50 can be prepared for comparison with the original 0.33:1 composition. The multihydric alcohol compound glycerol was then exposed to the catalyst composition at temperatures of 280° C., 300° C., and 320° C. Data for both catalysts are provided in Tables 14-19 below.

TABLE 14
Effect of metal-phosphate ratio on Co catalyst performance at 280° C.

| Co/ | Carbon | | Acrolein | | | Intrinsic |
|---|---|---|---|---|---|---|
| PO₄ | Balance | Conv. | Yield | Acrolein | Acetol | Acrolein |
| ratio | (%) | (%) | (%) | Selectivity | Selectivity | Selectivity |
| 0.33 | 98.46 | 97.45 | 85.03 | 0.87 | 0.11 | 0.884 |
| 0.5 | 97.32 | 99.5 | 86.16 | 0.87 | 0.11 | 0.894 |
| 0.67 | 89.96 | 88.31 | 68.94 | 0.78 | 0.11 | 0.867 |
| 0.75 | 87.16 | 58.8 | 40.42 | 0.69 | 0.09 | 0.792 |
| 1 | 93.29 | 23.61 | 14.66 | 0.62 | 0.09 | 0.665 |

TABLE 15
Effect of metal-phosphate ratio on Co catalyst performance at 300° C.

| | Carbon | | Acrolein | | Acetol | Intrinsic |
|---|---|---|---|---|---|---|
| Co/PO₄ | Balance | Conv. | Yield | Acrolein | Selec- | Acrolein |
| ratio | (%) | (%) | (%) | Selectivity | tivity | Selectivity |
| 0.33 | 96.79 | 99.6 | 82 | 0.82 | 0.14 | 0.847 |
| 0.5 | 104.21 | 99.82 | 92.21 | 0.92 | 0.12 | 0.883 |
| 0.67 | 94.96 | 98.87 | 81.23 | 0.82 | 0.13 | 0.864 |
| 0.75 | 98.33 | 84.91 | 72.61 | 0.86 | 0.13 | 0.875 |
| 1 | 92.84 | 39.23 | 27.5 | 0.7 | 0.12 | 0.754 |

TABLE 16
Effect of metal-phosphate ratio on Co catalyst performance at 320° C.

| | Carbon | | Acrolein | | Acetol | Intrinsic |
|---|---|---|---|---|---|---|
| Co/PO₄ | Balance | Conv. | Yield | Acrolein | Selec- | Acrolein |
| ratio | (%) | (%) | (%) | Selectivity | tivity | Selectivity |
| 0.33 | 100.88 | 99.61 | 83.9 | 0.84 | 0.17 | 0.833 |
| 0.5 | 100.4 | 99.76 | 87.15 | 0.87 | 0.13 | 0.867 |
| 0.67 | 104.46 | 99.55 | 88.79 | 0.89 | 0.15 | 0.852 |
| 0.75 | 98.39 | 96.39 | 81.02 | 0.84 | 0.14 | 0.854 |

TABLE 17
Effect of metal-phosphate ratio on Ba catalyst performance at 280° C.

| | Carbon | | Acrolein | | Acetol | Intrinsic |
|---|---|---|---|---|---|---|
| Ba/PO₄ | Balance | Conv. | Yield | Acrolein | Selec- | Acrolein |
| ratio | (%) | (%) | (%) | Selectivity | tivity | Selectivity |
| 0.33 | 98.67 | 99.78 | 84.28 | 0.845 | 0.142 | 0.856 |
| 0.5 | 49.51 | 92.56 | 36.79 | 0.397 | 0.057 | 0.803 |
| 0.75 | 44.87 | 74.88 | 17.20 | 0.230 | 0.034 | 0.512 |
| 1.0 | 52.93 | 0.00 | 0.00 | 0.000 | 0.000 | 0.000 |
| 1.5 | 55.12 | 0.00 | 0.00 | 0.000 | 0.000 | 0.000 |

TABLE 18
Effect of metal-phosphate ratio on Ba catalyst performance at 300° C.

| | Carbon | | Acrolein | | Acetol | Intrinsic |
|---|---|---|---|---|---|---|
| Ba/PO₄ | Balance | Conv. | Yield | Acrolein | Selec- | Acrolein |
| ratio | (%) | (%) | (%) | Selectivity | tivity | Selectivity |
| 0.33 | 74.29 | 93.46 | 59.56 | 0.637 | 0.088 | 0.858 |
| 0.5 | 48.25 | 87.56 | 31.49 | 0.360 | 0.049 | 0.745 |
| 0.75 | 49.51 | 66.25 | 13.72 | 0.207 | 0.031 | 0.418 |
| 1.0 | 60.30 | 0.00 | 0.00 | 0.000 | 0.000 | 0.000 |
| 1.5 | 57.42 | 0.00 | 0.00 | 0.000 | 0.000 | 0.000 |

TABLE 19
Effect of metal-phosphate ratio on Ba catalyst performance at 320° C.

| | Carbon | | Acrolein | | Acetol | Intrinsic |
|---|---|---|---|---|---|---|
| Ba/PO₄ | Balance | Conv. | Yield | Acrolein | Selec- | Acrolein |
| ratio | (%) | (%) | (%) | Selectivity | tivity | Selectivity |
| 0.33 | 103.62 | 99.85 | 88.42 | 0.89 | 0.15 | 0.855 |
| 0.5 | 46.58 | 79.20 | 22.16 | 0.280 | 0.046 | 0.601 |
| 1.5 | 56.12 | 0.00 | 0.00 | 0.000 | 0.000 | 0.000 |

Referring to Tables 14-19, it can be observed that for all compositions of Co and Ba, increasing temperature can lead to increased conversion, decreased acrolein selectivity, and increased acetol selectivity, as was observed with the parent 0.33:1 M:PO₄ ratio compositions. The effect of the metal:phosphate ratio at a particular temperature may depend on the metal employed. For cobalt catalysts, activity and selectivity diminished with an increasing Co:PO₄ ratio with the optimum ratio for both activity and selectivity observed at 0.5:1. This composition, assuming a statistically equilibrated mixture, can correspond to a bulk stoichiometry of Co(H₂PO₄)₂. For the barium catalysts, compositions with Ba:PO₄ ratios less than 1.0 can be active with the original 0.33:1 Ba:PO₄ composition providing the highest activity and selectivity. For compositional ratios of 1.0 and 1.5, bulk stoichiometries of BaHPO₄ and Ba₃(PO₄)₂, respectively, would result. With decreasing metal:phosphate ratios, increasing acidity results with dihydrogenphosphates representing bulk stoichiometries for divalent metals at ratios of 0.5 and mixtures of phosphoric acid and metal dihydrogenphosphate complexes at lesser ratios. Thus, while monohydrogenphosphate complexes of cobalt (Co:PO₄=1.0) may appear to contain enough acidity to effect glycerol dehydration, barium complexes require some degree of dihydrogenphosphate for effectiveness. Taking precipitation kinetics into account during catalyst preparation, while a divalent M:PO₄ ratio of 0.5 would correspond to a bulk stoichiometry of M(H₂PO₄)₂, the composition may in fact comprise equal quantities of H₃PO₄ and MHPO₄ when accounting for the low solubility of metal phosphate-salts. Therefore, unless equilibration of acid and basic sites occurs post-precipitation (e.g., during calcinations), it may be that working catalysts exhibit a distribution of compositions based on metal identity and stoichiometry.

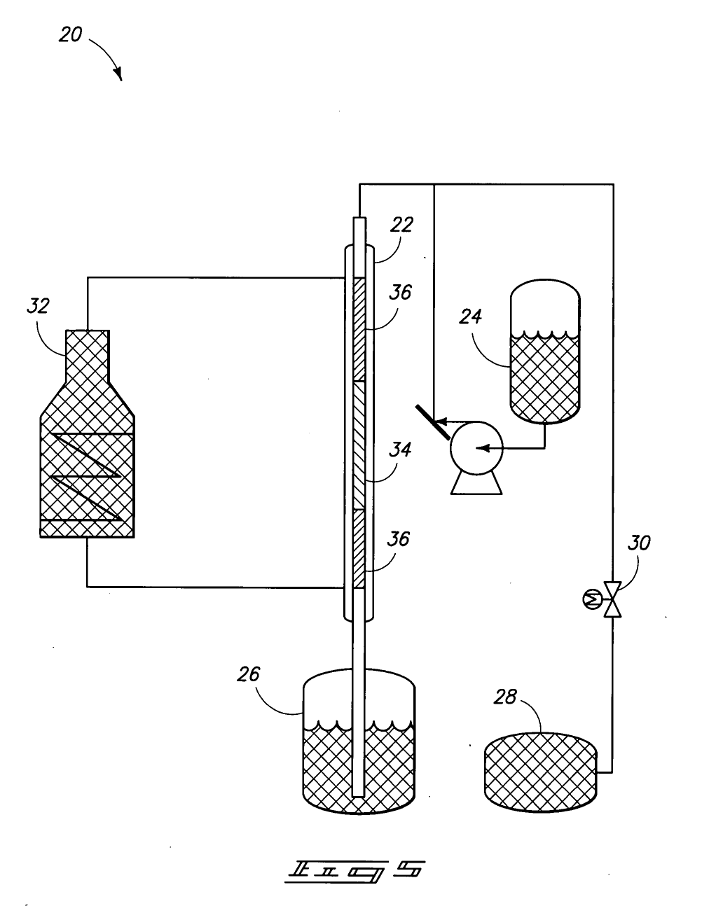

In accordance with another embodiment, FIG. 5 depicts a chemical production system 20 that includes a reactor 22 coupled to a reactant reservoir 24 as well as a product reservoir 26. Reactant of reactant reservoir 24 can be a multihydric alcohol compound, for example.

To facilitate the flow of reactant from reactant reservoir 24 to reactor 22, a carrier composition 28 including a gas or liquid such as nitrogen is provided to a reactant reservoir conduit utilizing flow control 30. In accordance with another embodiment, CO₂ can be utilized as the carrier composition 28. These solid support beds can also be treated with CO₂ and reactant from reactor reservoir 24 can be combined with carrier composition 28 and provided to reactor 22 thus changing the reactant composition prior to entering reactor 22.

The reactant composition can include both the multihydric compound and an inert compound, for example. As another example, the reactant composition can include one or more of water, nitrogen, and carbon dioxide. Nitrogen can be exposed to the catalyst at a rate of at least about 50 sccm or from about 50 sccm to about 100 sccm. Utilizing these reactant compositions, the catalyst composition may be maintained at a temperature of from about 200° C. to about 500° C.

Reactor 22 can be configured as an oil heated reactor utilizing an oil heater 32. Reactor 22 can be configured having a catalyst 34 supported by packing material 36. Packing material 36 can include quartz wool. In accordance with one example, glycerol from reactant reservoir 24 can be provided with carrier composition 28 to reactor 22 and exposed to catalyst 34 such as solid support materials. Prior to exposing reactant to catalyst 34, catalyst 34 can be exposed to carrier composition 28 such as CO₂. As an example, silica alumina material catalyst utilized in combination with CO₂ can facilitate a glycerol conversion more than double with increased acrolein yield.

Improved process consistency can occur by including a nitrogen sweep with reactant composition of glycerol/water, the producing a reactant composition being provided to the catalyst that includes glycerol, water, and nitrogen. The nitrogen feed can be small in comparison to the vaporized volumes of the liquid feed glycerol and water; the addition of an inert can have only minor impacts on the contact times of the vapor phase reactor, but can facilitate liquids condensing in the sample collection system. Alternative systems can rely on small cross section tubing to facilitate the liquid exiting the reactor being collected, with larger exit line tubing utilizing gravity draining of product material.

While residence times can be only minimally affected by inclusion of an inert gas feed, the sweep can effect product selectivity and recovery. For example, when MHPO₄—H₃PO₄ catalysts (M═Fe, MN) were evaluated at 300° C. using a standard feed rate of 12 mL/h of 30% aqueous glycerol, variations in acrolein selectivity can result (Table 20). For example, incrementally increasing the nitrogen sweep feed from 50 sccm to 75 sccm to 100 sccm can result in decreasing the contact-time from 250 milliseconds to 230 milliseconds to 210 milliseconds, a net change of less than 20%. However, with decreasing contact times, acrolein yields and selectivities can increase and then decrease passing through an apparent maximum. Such a result can be indicative of improved acrolein recovery.

TABLE 20
Effect of contact time on product selectivities for Co_{0.33}H_{2.33}PO₄

| | | | | Acrolein | | |
|---|---|---|---|---|---|---|
| | RT | N₂ flow | Conv. | Yield | Acrolein | Acetol |
| M | (sec) | (sccm) | (%) | (%) | Selectivity | Selectivity |
| Mn | 0.21 | 100.00 | 99.95 | 71.77 | 0.718 | 0.112 |
| Mn | 0.23 | 75.00 | 99.88 | 80.43 | 0.805 | 0.128 |
| Mn | 0.25 | 50.00 | 97.26 | 53.79 | 0.553 | 0.082 |
| Fe | 0.21 | 100.00 | 98.57 | 83.84 | 0.851 | 0.131 |
| Fe | 0.23 | 75.00 | 99.74 | 86.49 | 0.867 | 0.149 |
| Fe | 0.25 | 50.00 | 99.77 | 77.16 | 0.774 | 0.135 |

In compliance with the statute, this disclosure has been provided in language more or less specific as to structural and methodical features. It is to be understood, however, that the disclosure is not limited to the specific features shown and described, since the means herein disclosed comprise preferred forms of putting the invention into effect. The invention is, therefore, claimed in any of its forms or modifications within the proper scope of the appended claims appropriately interpreted in accordance with the doctrine of equivalents.

1. A chemical production process comprising exposing a reactant composition to a catalyst composition to form a product composition, wherein:
the reactant composition comprises glycerol;
the catalyst composition is effective to dehydrate at least a portion of the glycerol; and
the reactant composition is exposed to the catalyst composition for less than about 0.25 seconds.

2. The chemical production process of claim 1 wherein the reactant composition is exposed to the catalyst composition for less than about 0.14 seconds.

3. The chemical production process of claim 1 wherein the reactant composition is exposed to the catalyst composition for from about 0.07 seconds to about 0.25 seconds.

4. The chemical production process of claim 1 wherein;
the product composition comprises one or both of acrolein and acetol.

5. The chemical production process of claim 1 wherein the product composition is later utilized as a reactant.

6. The chemical production process of claim 1 wherein the catalyst composition comprises phosphorous or niobium.

7. The chemical production process of claim 1 wherein the catalyst composition is supported.

8. The chemical production process of claim 1 wherein the catalyst composition is supported with a silica support.

9. The chemical production process of claim 1 wherein the catalyst composition comprises sulfur.

10. The chemical production process of claim 1 wherein the product composition comprises both acrolein and acetol.

11. The chemical production process of claim 10 wherein every mole of glycerol exposed to the catalyst composition forms at least about 0.1 moles of product composition.

12. The chemical production process of claim 10 wherein every mole of glycerol exposed to the catalyst composition forms about 0.1 moles to about 0.99 moles of product composition.

13. The chemical production process of claim 10 wherein a ratio of acrolein to acetol is at least about 3:2.

14. The chemical production process of claim 13 wherein a ratio of acrolein to acetol is from about 3:2 to about 10:1.

15. A chemical production process comprising exposing a reactant composition to a catalyst composition to form a product composition, wherein:
the reactant composition comprises glycerol;
the catalyst composition is effective to dehydrate at least a portion of the glycerol;
the product composition comprises a carbonyl compound; and
the catalyst is maintained at a temperature of from about 280° C. to about 320° C.

16. The chemical production process of claim 15 wherein:
the product composition comprises one or both of acrolein and acetol.

17. The chemical production process of claim 15 wherein the product composition is later utilized as a reactant.

18. The chemical production process of claim 15 wherein the catalyst composition comprises phosphorous or niobium.

19. The chemical production process of claim 15 wherein the catalyst composition comprises sulfur.

20. The chemical production process of claim 15 wherein the product composition comprises both acrolein and acetol.

21. The chemical production process of claim 20 wherein every mole of glycerol exposed to the catalyst composition forms at least about 0.1 moles of product composition.

22. The chemical production process of claim 20 wherein every mole of glycerol exposed to the catalyst composition forms about 0.1 moles to about 0.99 moles of product composition.

23. The chemical production process of claim 20 wherein a ratio of acrolein to acetol is at least about 3:2.

24. The chemical production process of claim 23 wherein a ratio of acrolein to acetol is from about 3:2 to about 10:1.

25. A chemical production process comprising exposing a reactant composition to a catalyst composition to form a product composition, wherein:
the reactant composition comprises glycerol and an inert compound;
the catalyst composition is effective to dehydrate at least a portion of the glycerol; and
the product composition comprises a carbonyl compound.

26. The chemical production process of claim 25 wherein;
the product composition comprises one or both of acrolein and acetol.

27. The chemical production process of claim 25 wherein the catalyst composition comprises phosphorous or niobium.

28. The chemical production process of claim 25 wherein the catalyst composition is supported.

29. The chemical production process of claim 25 wherein the catalyst composition is supported with a silica support.

30. The chemical production process of claim 25 wherein the catalyst composition comprises sulfur.

31. The chemical production process of claim 25 wherein the catalyst composition is maintained at a temperature of from about 200° C. to about 500° C.

32. The chemical production process of claim 25 wherein the reactant composition comprises the glycerol and one or more of water, nitrogen, and carbon dioxide.

33. The chemical production process of claim 25 wherein the reactant composition comprises the glycerol and water.

34. The chemical production process of claim 33 wherein the glycerol is at least 17% (wt./wt.) of the reactant composition.

35. The chemical production process of claim 33 wherein the glycerol is from about 17% (wt./wt.) to about 30% (wt./wt.) of the reactant composition.

36. The chemical production process of claim 25 wherein the reactant composition comprises the glycerol and nitrogen, the nitrogen being exposed to the catalyst composition at a rate of at least 50 sccm.

37. The chemical production process of claim 25 wherein the reactant composition comprises the glycerol and nitrogen, the nitrogen being exposed to the catalyst composition at a rate of from about 50 sccm to about 100 sccm.
